(19) United States
(12) Plant Patent Application Publication
(10) Pub. No.: US 2002/0138888 P1
(43) Pub. Date: Sep. 26, 2002

(54) Gladiolus hybrid plant named 'Tushar Mauli'

(76) Inventors: Devashish Mukhrjee, Himachal Pradesh (IN); Devendra Dhayani, Himachal Prades (IN); Jaichand Rana, Himacharl Pradesh (IN)

Correspondence Address: CHRISTIE, PARKER & HALE, LLP, 350 WEST COLORADO BOULEVARD, SUITE 500, PASADENA, CA, 91105, US

(21) Appl. No.: 09/815,396

(22) Filed: Mar. 22, 2001

(51) Int. Cl. A01H 5/00

(52) U.S. Cl. PLT/301

(57) ABSTRACT

The invention relates to a new and distinct Gladiolus hybrid plant named ‘Tushar Mauli’ characterized by its attractive Dutch Vermilion colored flowers with Dresden yellow color on the center of the lip petals.

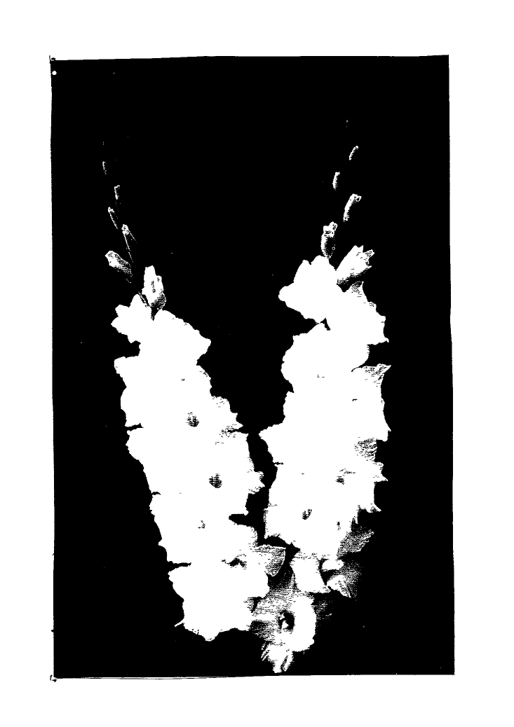

### BACKGROUND OF THE INVENTION

The present invention relates to a new and distinct variety of Gladiolus named ‘TUSHAR MAULI’, a member of the Gladiolus genus. The new variety is the hybrid between the Gladiolus plants “Bonfire” and “Eurovision” (Hybrid varieties).

Gladiolus is one of the important cut flowers throughout the world. The commercial cultivation is wide spread in temperate, tropical and subtropical climates. The demand of new varieties with better color, quality flowers, and planting materials is always existing and rising in the floriculture trade.

Applicants initiated a breeding program to develop better types of Gladiolus hybrids suitable to wide range of climatic conditions, and having wide range of characteristics such as better color, increased number of florets and spike length as per the international standards, better yield of corm and cormels, tolerant to the common diseases etc.

Accordingly, applicants initiated a breeding program involving hybridization of commonly available Gladiolus plants in the fields at Institute of Himalayan Bioresource Technology, Palampur, India, in order to develop new and distinct varieties of Gladiolus plants. In other words, the hybrids were developed by crossing parental genotypes involving asexual hybridization in the breeding program.

The program yielded a number of hybrid plants out of which one genotype namely IHBT-GH-381 was selected and named ‘TUSHAR MAULI’. This plant was found to have new color, flower size, number of florets per spikes, length of flower spikes, better yield of corms and cormels and less prone to common diseases. Growing the plant on a commercial scale offers the horticulturists an improved and new variety, which can be commercially cultivated.

### FIELD OF THE INVENTION

The present invention relates to a new Gladiolus hybrid plant named ‘Tushar Mauli’ and belonging to the family Iridaceae. The new variety is propagated vegetatively by corms and hence can be maintained as a stable genotype. The plant of the new variety is an ornamental plant widely cultivated for its beautiful flowers which are of commercial and export value.

### SUMMARY OF THE INVENTION

The present invention provides a new and distinct hybrid Gladiolus plant named ‘TUSHAR MAULI’ characterized by its attractive standard type white color flowers with Neyron Rose edges having a greenish color throat with purple tinge and ruffled petals.

### BRIEF DESCRIPTION OF ILLUSTRATION

The accompanying photographic illustration shows a typical specimen of the new variety, of field grown flower spike of IHBT-GH-381 (‘TUSHAR MAULI’)depicting standard type flowers of White (near 158C) with Neyron Rose (near 56A) edges and having a greenish with purple (near 80D) colored tinge.

### DESCRIPTION OF THE NEW VARIETY

The new variety provides a new and distinct Gladiolus genotype named as ‘TUSHAR MAULI’. This plant has been developed through planned breeding experiments conducted at Institute of Himalayan Resources, (IHBT) Palampur, Himachal Pradesh, India with defined aim to develop superior Gladiolus genotypes. For this purpose, Gladiolus varieties were collected from different sources and grown in the fields at Palampur, India for facilitating breeding program. The emasculation and pollination in different varieties were carried out during the months of April and May 1991. The seeds were collected between July and August 1991 and sown in beds under open field conditions and covered with dry grasses in about December 1991. The resultant seedlings were space planted in the field at Palampur in March and April 1992. The corm and cormels of survived hybrid plants were replanted continuously four years for screening and multiplication.

Based on superior performance for attractive color combination, compactness of flower spikes, number of flowers per spikes, length of flower spikes, number of flowers remains open at a time, number of corm and cormel production per plant, the plant of this invention (IHBT-GH-381) was selected for further observation and evaluation.

Considering the superior characteristics like excellent color, number of flowers, compactness of flower spikes, plant height, ruffled-ness of flower petals, regeneration potential and freedom from common diseases, it was asexually reproduced through corms and cormels to maintain purity.

The selected hybrid IHBT-GH-381 was named ‘TUSHAR MAULI’ and grown at row distance of about 1 feet and plant to plant about 6 inches for four consecutive years to study its growth and flowering performance and multiplication. Data was recorded on randomly selected twenty plants every year. The hybrid IHBT-GH-381 maintained uniformity in its growth and flowering performance.

### Evidence of Uniformity and Stability

The new variety has remained stable and uniform for its morphological characters and showed consistency in performance for various growth and flowering parameters during evaluation and vegetative multiplication since 1992. Throughout the evaluation period of the new variety no variants were found from the normal population.

### OBJECTIVE DESCRIPTION OF THE GENOTYPE ‘TUSHAR MAULI’

The following is an objective description of the new variety.

Genus: Gladiolus

Species: Hybrid

Family: Iridacae

Common name: Sward lily/Gladiolus

Average plant height: About 159.84 cm.

Growth habit: Erect, uniform

Average stem diameter: About 1.41 cm.

Average number of leaves/plant: About 7.98

Average height of leaves: About 70.01 cm.

Average days to flower: About 101 days

Type of flowers: Standard

Average number of spikes/plant: About 1.42

Average length of the flower spike: About 141 cm.

Average number of flowers/spike: About 17.90

Flower color: White near 158C, with Neyron Rose near 56A edges having a greenish with purple near 80D tinge colored throat

Type of petals: Ruffled

Average number of flowers remains open at a time: About 7.44

Average longevity of the 1st flower: About 3.52 days

Average diameter of the 1st flower: About 13.3 cm.

Average longevity of the spike: About 11.8 days

Average number of corms/plant: About 1.55

Average diameter of corms: About 6.77 cm.

Average Number of cormels/plant: About 239

We claim:

1. A new and distinct Gladiolus plant named ‘TUSHAR MAULI’ substantially as herein described and illustrated.
